(19) United States
(12) Patent Application Publication
(10) Pub. No.: US 2007/0233159 A1
(43) Pub. Date: Oct. 4, 2007

(54) DEPILATING DEVICE

(75) Inventors: Hidekazu Sueyoshi, Higashiomi (JP); Wei Wang, Hikone (JP); Tetsuro Hashiguchi, Hikone (JP); Shinji Nishimura, Hikone (JP)

Correspondence Address: GREENBLUM & BERNSTEIN, P.L.C., 1950 ROLAND CLARKE PLACE, RESTON, VA, 20191, US

(73) Assignee: MATSUSHITA ELECTRIC WORKS, LTD., Osaka (JP)

(21) Appl. No.: 11/691,591

(22) Filed: Mar. 27, 2007

(30) Foreign Application Priority Data
Mar. 28, 2006 (JP) 2006-089576

(51) Int. Cl. A61B 17/50

(52) U.S. Cl. 606/133

(57) ABSTRACT

In a depilating device for making depilation using a soap in which a depilation head including a depilating portion for removing hair is attached to a main body casing including a manually grippable grip and having a waterproof structure, a wiper for wiping off bubbles adhering to a skin surface after depilating is provided on a head frame that surrounds the depilating portion.

### CROSS REFERENCE TO RELATED APPLICATION

This application is based upon and claims the benefit of priority from prior Japanese Patent Application P2006-089576, filed on Mar. 28, 2006; the entire contents of which are incorporated by reference herein.

### BACKGROUND OF THE INVENTION

1. Field of the Invention

The present invention relates to a depilating device capable of removing hair using soap.

2. Description of the Related Art

Conventionally, there has been proposed a depilating device configured so that a depilating unit is attached to a main body casing including a driving source. As a depilating device of this type, there has been known a depilating device in which a depilating unit includes a rotary cylinder rotated by a driving source and a depilation head having a plurality of depilating claws driven to be opened/closed by rotation of the rotary cylinder and used to catch and remove hair (for example, see Japanese Patent Application Laid-open No. 2001-149135).

Meanwhile, to reduce friction between skin surface and the rotary cylinder when depilating, depilation is often made using soap. In this case, the use of the soap can ensure safe depilation with respect to the skin surface; however, the main body casing needs to have a water proof structure. According to the conventional technique disclosed in Japanese Patent Application Laid-Open No. 2001-149135, a water proof member is provided halfway through a drive transmitter that transmits driving to the rotary cylinder from the driving source within the main body casing, thereby constituting the waterproof structure between the driving source and the rotary cylinder.

However, the depilation using soap has the following disadvantages. When the hair is removed using the soap, soap bubbles spread by the rotation of the rotary cylinder and the skin surface after the depilation becomes invisible by the bubbles. This makes it difficult to discriminate whether unremoved hairs remain. Due to this, a user is forced to wipe off the bubbles by himself or herself whenever the skin surface becomes invisible, which takes lots of trouble and deteriorates user-friendliness of the depilating device.

### SUMMARY OF THE INVENTION

It is an object of the present invention to provide a depilating device capable of preventing unremoved hairs from becoming invisible by soap bubbles adhering to a skin surface of a user at the time of depilation using soap, capable of saving time and labor for wiping off the bubbles, and capable of improving its user-friendliness.

According to one aspect of the present invention, there is provided a depilating device comprising: a mainbody casing including a manually grippable grip and having a waterproof structure; a depilation head including a depilating portion for removing hair and a head frame surrounding the depilating portion, the depilation head attached to the main body casing; and a wiper provided on the head frame, and wiping off a soap bubble adhering to a skin surface after depilation by the depilating portion.

By so configuring, when the depilation head is moved to make depilation using soap, the wiper is moved to follow the depilating portion. Due to this, even if soap bubbles spread when depilating, the wiper wipes off the bubbles adhering to the skin surface after depilating. This can prevent the skin surface in a depilation region from becoming invisible by the bubbles, thereby making is possible to instantly recognize unremoved hairs.

Furthermore, it is preferable that the wiper is plate-shaped to extend almost in parallel to the skin surface while the depilation head is pressed against the skin surface. In this case, the plate-shaped wiper can completely wipe off the soap bubbles present in the depilation region. It is, therefore, possible to more clearly recognize unremoved hairs and further improve the user-friendliness of the depilating device.

Moreover, it is preferable that the wiper is deformable in a directional most perpendicular to the skin surface. In this case, the follow-up performance of the wiper even with respect to irregular parts of the skin surface can be improved. It is thereby possible to ensure wiping off soap bubbles on whichever part of the skin surface.

It is further preferable that the wiper is floatable on the skin surface. In this case, the floatable wiper makes it possible to eliminate the stimulus on the skin, and the follow-up performance of the wiper even with respect to such a convex part as a bony part of the skin surface can be improved.

It is also preferable that the wiper is provided on one of a front side and a back side of the main body casing. In this case, by arranging the front side (or back side) of the main body casing, on which side the wiper is provided, in rear of the depilation head while a front surface and a back surface of the main body casing are being gripped by user's fingers, the wiper can ensure wiping off the bubbles adhering to the skin surface after depilating using the depilation head.

Furthermore, it is preferable that the wiper and the head frame are detachable from the main body casing. In this case, by detaching the wiper as well as the head frame from the main body casing, it is possible to easily remove flocks adhering to the wiper and the head frame or those entering into the depilation head, thus facilitating cleaning operation.

Moreover, it is preferable that the wiper is detachable from the head frame. In this case, if dry depilation is made in a dry state where soap is not used, the wiper can be detached from the head frame. This can facilitate switching depilation operation between depilation using soap and dry depilation and thereby improving the user-friendliness of the depilating device. This can also facilitate replacement operation when the wiper deteriorates.

Further, it is preferable that the wiper is height-adjustable with respect to the head frame in a direction almost perpendicular to the skin surface. In this case, the height of the wiper can be switched with respect to a direction almost perpendicular to the skin surface. Therefore, if the depilation is made using soap, the wiper is lifted and held at a position at which the wiper touches the skin surface. At this time, by vertically adjusting the wiper, it is possible to easily adjust pressing load of the wiper on the skin surface, whereby the stimulus can be made weaker. Besides, it is possible to remove hair while stretching the skin using the wiper by the appropriate pressing load of the wiper on the skin surface. On the other hand, if the depilating device is used without soap, the wiper can be lowered and held at a position at which the wiper does not touch the skin. It is, therefore, possible to easily deal with dry depilation.

Furthermore, it is preferable that the wiper is made of elastomer. In this case, it is possible to eliminate stimulus on the skin by using the elastomer wiper and prevent damage on the skin surface. Besides, it is possible to sufficiently improve the follow-up performance of the wiper even with respect to such a convex part as a bony part of the skin surface.

### BRIEF DESCRIPTION OF THE DRAWINGS

FIG. 1 is a perspective view of a depilating device including a wiper according to a first embodiment of the present invention;

FIG. 2 is a cross-sectional front view of the depilating device according to the first embodiment;

FIG. 3 is a cross-sectional side view of the depilating device according to the first embodiment;

FIG. 4 is a side view of the depilating device for explaining operation for making depilation using the depilating device according to the first embodiment;

FIG. 5 is a perspective view of a head frame including the wiper according to the first embodiment;

FIG. 6 is an exploded perspective view of the head frame according to the first embodiment;

FIG. 7 is a cross-sectional view of a depilating unit including a button for attaching or detaching a depilation head according to the first embodiment;

FIG. 8 is an exploded perspective view of a depilating device according to a second embodiment of the present invention;

FIG. 9 is an exploded perspective view of a depilating device according to a third embodiment of the present invention; and

FIG. 10 is a perspective view of a depilating device according to a fourth embodiment of the present invention.

### DETAILED DESCRIPTION OF THE PREFERRED EMBODIMENTS

Embodiments of the present invention will be explained below with reference to the accompanying drawings.

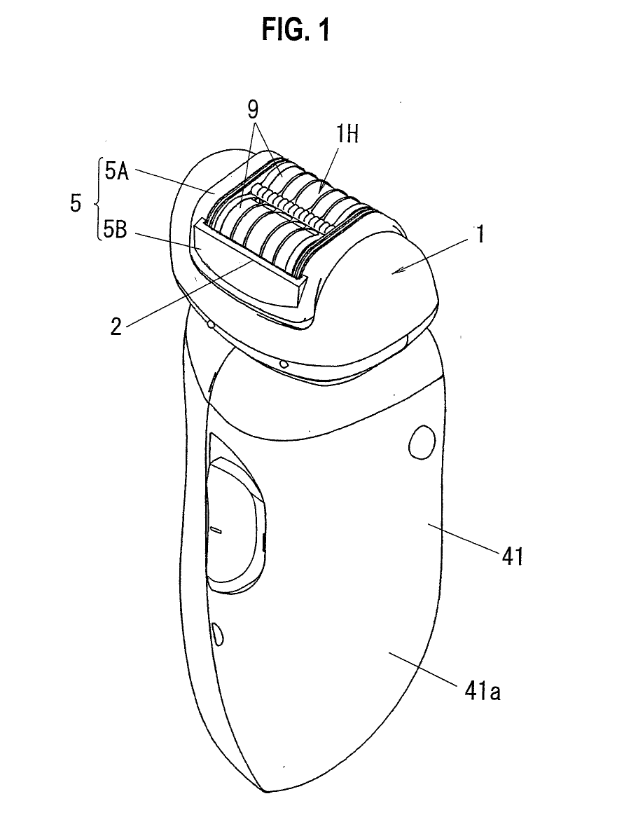

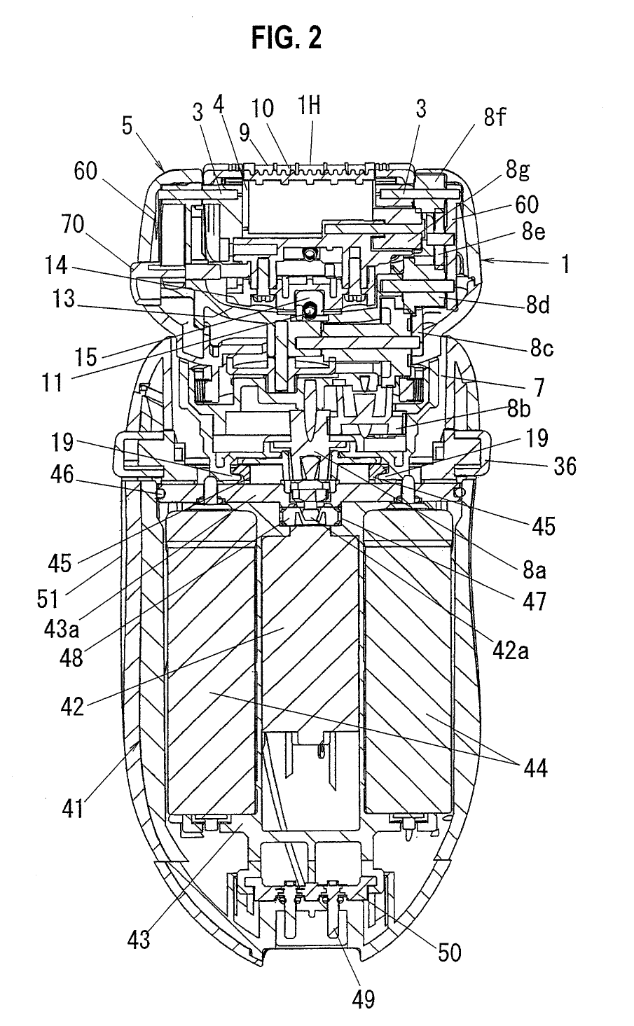

A depilating device according to a first embodiment of the present invention is a depilating device employed to remove hair from the body of a user for cosmetic purposes. As shown in FIGS. 1 and 2, the depilating device includes a main body casing 41 that includes a grip 41a which the user can grip by the hand and that includes therein a motor (driving unit) 42, and a depilating unit 1 that includes a depilation head 1H and that is detachably provided on the main body casing 41. The depilation head 1H includes rotary cylinders 4 and a depilating portion 9 having a plurality of depilating claws (e.g., a movable claw and a fixed claw) driven to be opened or closed by the rotary cylinders 4 and holding the hair between the depilating claws. The depilation head 1H is configured so that the hair is put between the depilating claws to remove the hair according to axial rotation of the rotary cylinders 4. The well-known technique disclosed in, for example, Japanese Patent Application Laid-Open No. 2001-149135 is applicable to a structure for driving the depilating claws to be open or closed by the rotation of the rotary cylinders 4. The structure is not, therefore, described in detail.

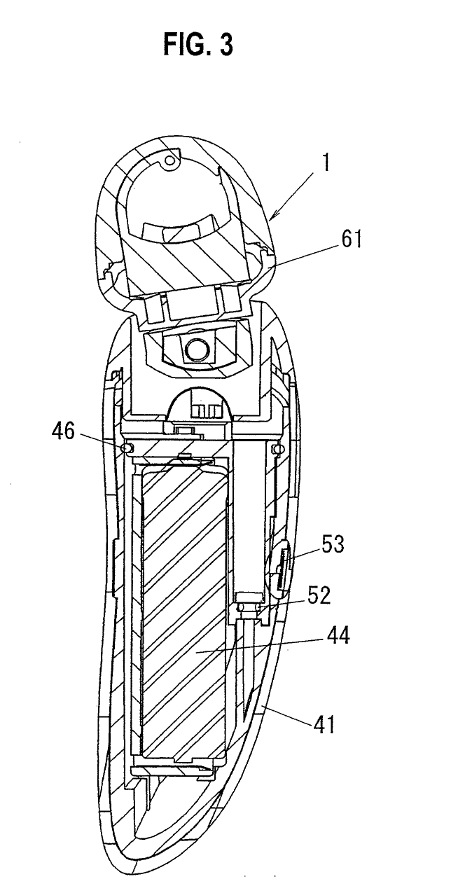

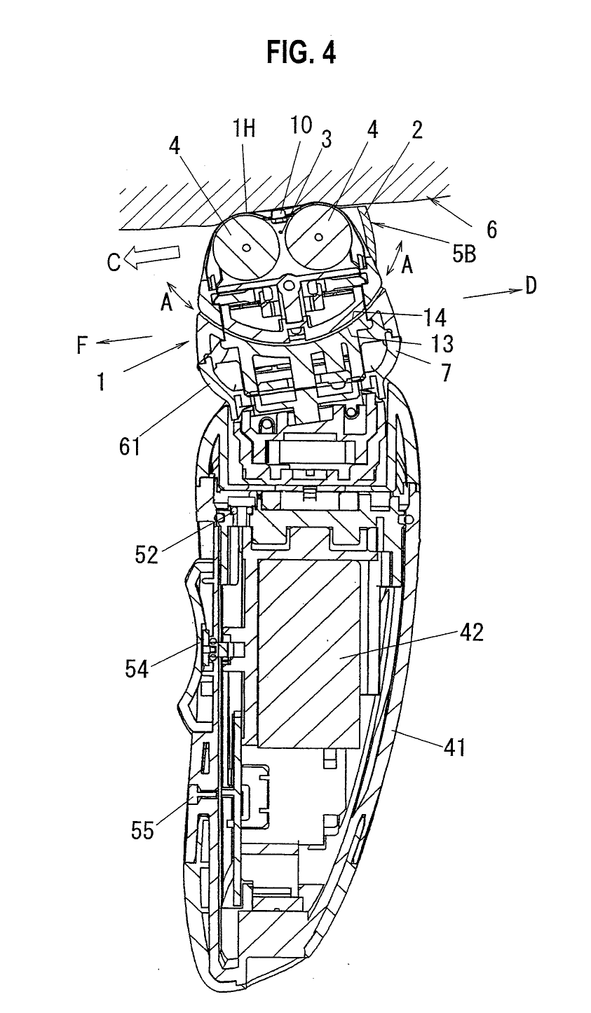

The main body casing 41 has a waterproof structure. As shown in FIG. 2, the main body casing 41 is configured so that two casings divided by half back and forth are cohered together, and an upper end of the main body casing 41 is opened. A motor 42, a base 43 including therein a gear train serving as a drive transmitter, and the like are incorporated in the main body casing 41. Furthermore, battery cells or chargers 44 for driving the motor 42 are incorporated in the main body casing 41. An upper end 43a of the base 43 closes an upper-end opening of the main body casing 41, hook attachments 45 are provided on an upper surface of the base 43, and a pair of hooks 19 is provided on a lower surface of the depilating unit 1. By detachably attaching the hooks 19 to the hook attachments 45, the depilating unit 1 can be engaged with the main body casing 41 and the depilating unit 1 can be disengaged from the main body casing 41 using an attachment/detachment unit 36. An outer shape of the upper end 43a of the base 43 is almost identical to that of the upper-end opening of the main body casing 41, and a first waterproof member 46, which is ring-shaped and elastic, is fitted into an outer circumference of the base 43. Moreover, an opening 47 into which an output shaft 42a of the motor 42 is penetrated is provided at the center of the upper end 43a of the base 43, and a second waterproof member 48, which has an M-shaped cross section, is fitted into the opening 47, thereby preventing entry of water into motor 42-side from outside. In FIG. 2, reference symbol 49 denotes a plug terminal pin, 50 denotes a plug base, and 51 denotes abattery-terminal O-ring. In FIGS. 3 and 4, reference symbol 52 denotes a waterproof-screw O-ring, 53 denotes a filter, 54 denotes a switch O-ring, and 55 denotes a lamp cover.

The depilating unit 1 is constituted by the depilation head 1H that integrally includes the two cylindrical rotary cylinders 4 for driving the depilating portion 9 to remove hear, a gear train 8a to 8g serving as a drive transmitter for transmitting rotation output of the motor 42 to the rotary cylinders 4, and a main body-side attachment 7 detachably attached to the main body casing 41.

As shown in FIG. 4, one rotating fulcrum 3 is provided to correspond to the two rotary cylinders 4 in the depilation head 1H, whereby the depilation head 1H is held rotatable around the rotating fulcrum 3. In the first embodiment, the rotating fulcrum 3 of the depilation head 1H is arranged at an intermediate position between the two rotary cylinders 4, thus structuring the two rotary cylinders 4 to always closely touch a skin surface 6 (see FIG. 4). Furthermore, in the first embodiment, the rotation of the rotary cylinders 4 is driven by rotation of the motor 42 within the main body casing 41. That is, a rotational movement generated by the motor 42 is transmitted to the two respective rotary cylinders 4 via the gear train 8a to 8g shown in FIG. 2.

As shown in FIG. 4, a lower end surface of the depilation head 1H is a convex curve 13 protruding arcuately toward the main body-side attachment 7, and an upper end surface of the main body-side attachment 7 is a concave curve 14 into which the convex curve 13 is fitted. While the convex curve 13 and the concave curve 14 are slidably abutted against each other, the rotating fulcrum 3 of the depilation head 1H is axially supported with respect to a pair of left and right holding pieces 60 of the main body-side attachment 7. The depilation head 1H is thereby slidably held with respect to the main body-side attachment 7 around the rotating fulcrum 3 in a direction indicated by an arrow A shown in FIG. 4. Furthermore, a coil spring 11 (see FIG. 2) for biasing the depilation head 1H toward a predetermined position (which is a central position in the first embodiment) in a rotating direction A is arranged on surfaces of the convex curve 13 and the concave curve 14 on which surfaces the concave curve 13 is opposed to the concave curve 14. By arranging the coil spring 11, as shown in FIG. 4, when the depilation head 1H is pushed against the skin surface 6 while the two rotary cylinders 4 are rotating, the depilation head 1H can rotate around one rotating fulcrum 3 according to irregularities of the skin surface 6, and the depilating claws rotate together with the depilation head 1H. It is thereby possible to efficiently remove many hairs using the two rotary cylinders 4. Moreover, even if the depilation head 1H entirely rotates according to the irregularities of the skin surface 6, depilation positions of the rotary cylinders 4 do not change. It is, therefore, possible to remove the hair less stimulatively to there by prevent the user's skin from being damaged. At the same time, a position at which the hair is put between the depilating claws is made closer to the skin surface 6. Therefore, each of the depilating claws can catch the root of the hair and continuously remove the hair, and the depilation operation can facilitate putting particularly a short hair between the depilating claws and removing it and can weaken depilation-caused stimulus.

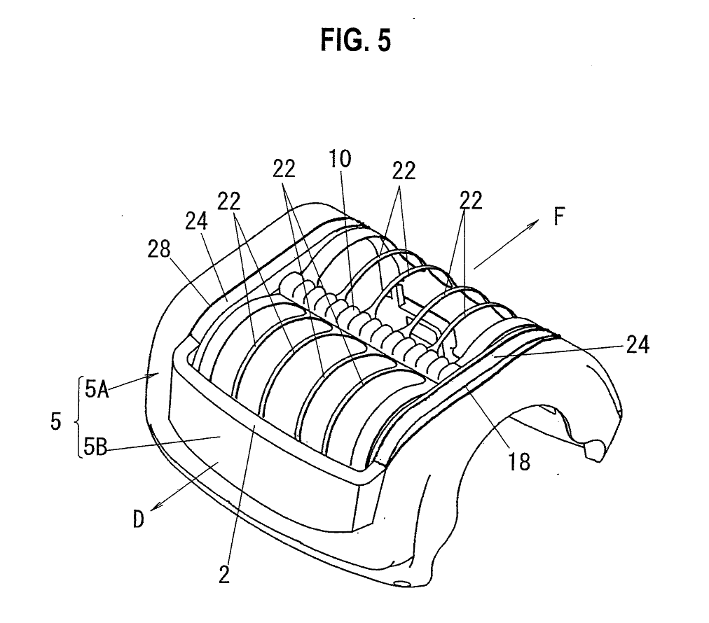

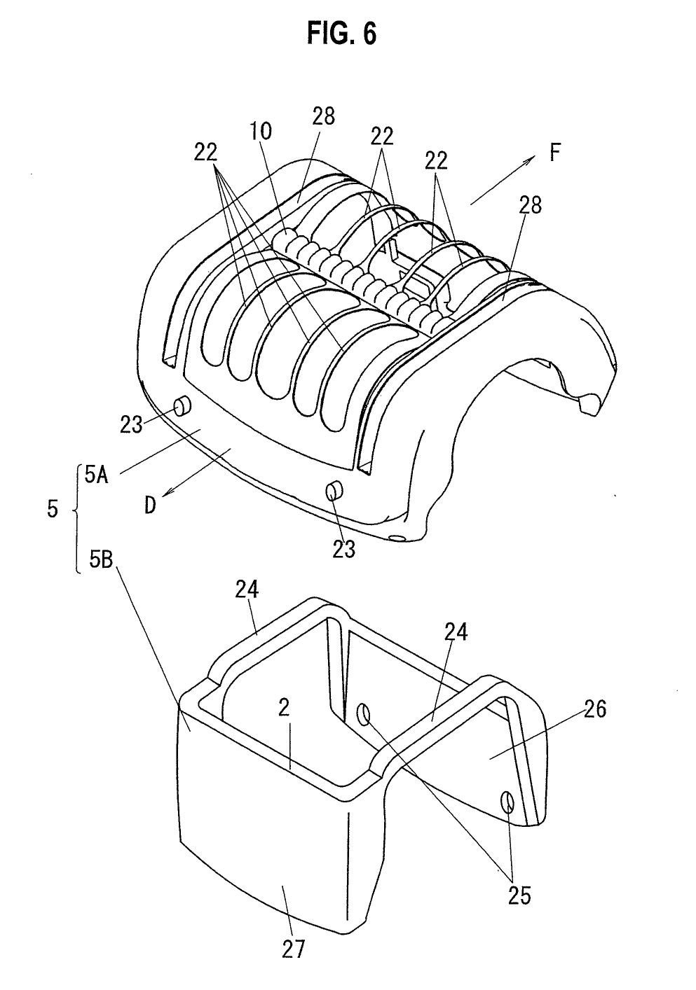

According to the first embodiment of the present invention, the depilation head 1H includes a head frame 5 provided to surround the depilating portion 9. The head frame 5, which is formed into a generally tubular shape, is constituted by an inner frame 5A for exposing an upper side of each of the rotary cylinders 4 from its upper opening and an outer frame 5B including a wiper 2 as shown in FIGS. 5 and 6.

A plurality of wires 22 is provided to stride over the upper opening of the inner frame 5A. The plural wires 22 are arranged along a horizontal direction (which is a direction of rotational axes of the rotary cylinders 4) so as not to obstruct rotational movements of the rotary cylinders 4. Each of the wires 22 is made of an elastic material. When the rotary cylinders 4 are pushed against the skin surface 6 to remove hair, the elastic wires 22 are bent and put between the skin surface 6 and the rotary cylinders 4. This makes the skin surface 6 separated from external exposed surfaces of the rotary cylinders 4 by as much as a diameter of each wire 22 in a portion in which the wires 22 are present between the skin surface 6 and the rotary cylinders 4. Accordingly, the skin surface 6 is not in direct contact with the external exposed surfaces of the rotary cylinders 4, thereby preventing damage on the skin. A comb 10 is provided in central recessed portions of the wires 22. By causing the comb 10 to strike against the skin, the rotary cylinders 4 can more smoothly slide on the skin.

Fitting slits 28 are provided on left and right sides of an upper portion of the inner frame 5A, respectively, and a plurality of frame attachment/detachment convex portions 23 is provided on each of front and back sides of a lower portion of the inner frame 5A. Fitting bridges 24 are provided on left and right sides of an upper portion of the outer frame 5B, respectively, and a plurality of frame attachment/detachment concave portions 25 is provided on each of front and back sides of a lower portion of the outer frame 5B. If the inner frame 5A is fitted into the outer frame 5B, then the fitting bridges 24 of the outer frame 5B are fitted into the respective fitting slits 28 of the inner frame 5A, and the frame attachment/detachment convex portions 23 of the outer frame 5B are fitted into the respective frame attachment/detachment concave portions 25 of the inner frame 5A as shown in FIG. 5. The inner frame 5A is thereby detachably attached to the outer frame 5B.

The wiper 2 for wiping off soap bubbles adhering to the skin surface 6 after depilating is provided on a front side F (which can be replaced by a back side D) of the outer frame 5B. The wiper 2 according to the first embodiment is formed into a plate shape extending almost in parallel to the skin surface 6 in a state where the depilation head 1H is pushed against the skin surface 6. Furthermore, as shown in FIG. 1, an upper end edge of the wiper 2 is arranged to be almost equal in width and height to the depilation head 1H. The wiper 2 is made of, for example, elastomer. Preferable examples of the elastomer include styrene elastomer and olefin elastomer. Needless to say, the material of the wiper 2 is not limited to the elastomer but can be, for example, natural rubber or soft foaming material.

When the depilation head 1H is moved in a direction indicated by an arrow C shown in FIG. 4 to remove hair using soap, the wiper 2 is moved to follow the depilation head 1H. Due to this, even if soap bubbles spread by the rotation of the rotary cylinders 4, the wiper 2 wipes off the bubbles adhering to the skin surface 6 simultaneously with removal of hair. This can prevent the skin surface 6 after depilating from becoming invisible by the bubbles, thereby making it possible to instantly recognize unremoved hairs. Moreover, the use of the soap can weaken the friction between the skin surface 6 and the rotary cylinders 4, thereby making it possible to ensure safe and smooth depilation with respect to the skin surface 6. Besides, because of its plate shape, the wiper 2 according to the first embodiment can completely wipe off the soap bubbles present in a depilation region, thereby making it possible to more clearly recognize the unremoved hairs. Further, because the wiper 2 is made of elastomer, it is possible to eliminate stimulus of the wiper 2 against the skin to prevent damage on the skin surface 6, and to sufficiently enhance follow-up performance of the wiper 2 even with respect to a convex part such as a bony part of the skin surface.

Moreover, the wiper 2 according to the first embodiment is provided on the back side D (which can be replaced by the front side F) of the main body casing 41. Therefore, by moving the depilation head 1H with the front side F of the main body casing 41 put forward while a front surface and a back surface of the main body casing 41 are being gripped by user's fingers as shown in FIG. 4, the wiper 2 can ensure wiping away the bubbles adhering to the skin surface 6 after depilating using the depilation head 1H. It is thereby possible to further enhance the user-friendliness of the depilating device according to the first embodiment.

Furthermore, according to the first embodiment, as shown in FIG. 6, the outer frame 5B including the wiper 2 is detachably attached to the inner frame 5A, thereby making it possible to facilitate replacement operation when the wiper 2 deteriorates.

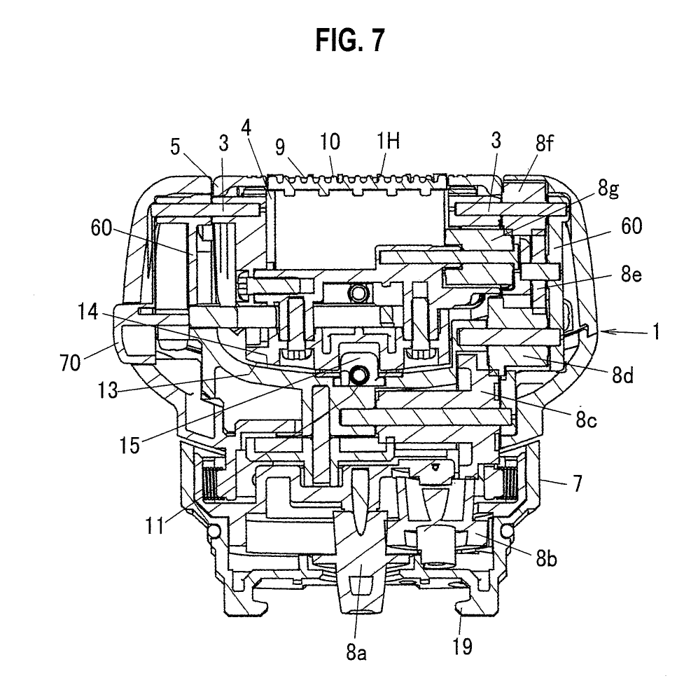

Moreover, the wiper 2 and the head frame 5 are detachably attached to the main body casing 41. In the first embodiment, a button 70 is provided on a left side surface of the depilating unit 1 as shown in FIG. 7. By pressing this button 70, a hook (not shown) of the depilating unit 1 is detached from the head frame 5 to make it possible to detach the head frame 5 as well as the wiper 2 from the main body casing 41. It is thereby possible to easily remove flocks adhering to the wiper 2 or the head frame 5 or those entering into the rotary cylinders 4 in the depilation head 1H, thus facilitating cleaning operation.

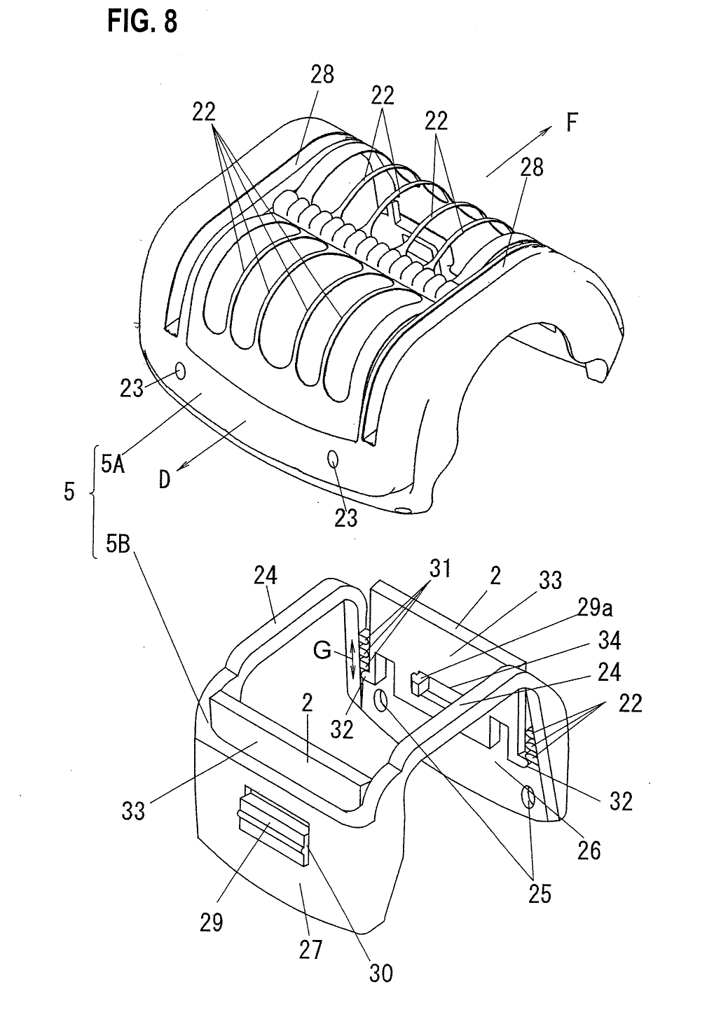

FIG. 8 shows an instance where the height of the wiper 2 can be adjusted relative to the head frame 5 in a direction almost perpendicular to the skin surface in a depilating device according to a second embodiment of the present invention. Other constituent elements of the depilating device according to the second embodiment shown in FIG. 8 are similar to those according to the first embodiment shown in FIGS. 5 and 6. In FIG. 8, like or corresponding constituent elements are denoted by the same reference symbols as those according to the first embodiment. In the second embodiment, a lifting plate 33 including the wiper 2 is slidable on the outer frame 5B in the vertical direction G. A pair of elastic hooks 32 is provided on lower left and right ends of the lifting plate 33, respectively. A plurality of vertical steps of engagement protrusions 31 is provided symmetrically on left and right ends of the outer frame 5B, respectively. Each of the hooks 32 is designed to be engaged with one of the vertical steps of engagement protrusions 31.

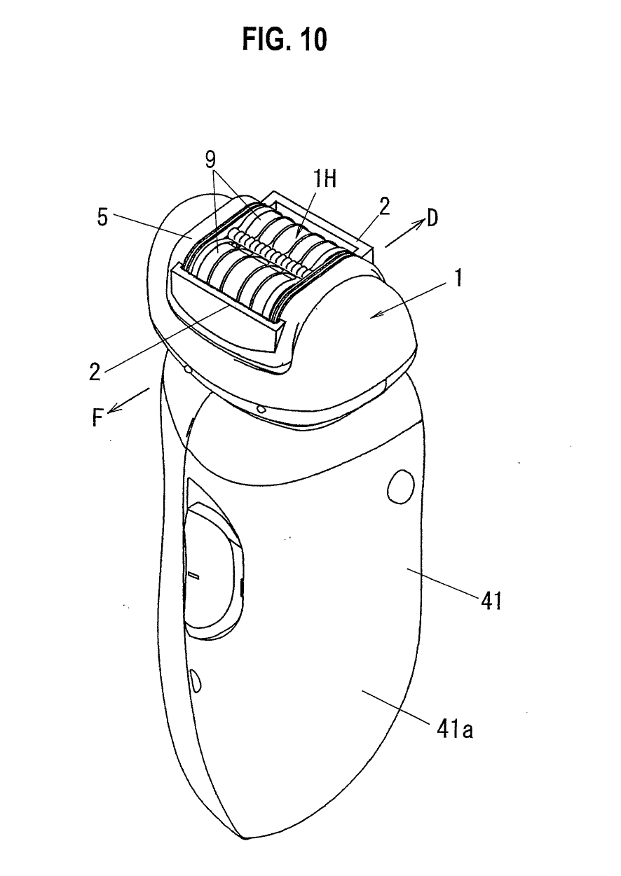

A height switching button 29 is provided on an outer surface of the lifting plate 33. A hook 29a is provided on a rear surface of the height switching button 29 to protrude therefrom. This hook 29a is engaged with a lateral bore 34 formed in the lifting plate 33, whereby the height switching button 29 is fixed to the lifting plate 33. A lateral hole 30 is formed in the outer frame 5B. The height switching button 29 protrudes from the lateral hole 30 toward outside of the outer frame 5B. When the user slides the height switching button 29 upward by the fingers, each of the hooks 32 is sequentially engaged with the vertical steps of engagement protrusions 31 from bottom up, whereby the height of the wiper 2 can be adjusted. FIG. 8 shows an instance (corresponding to FIG. 10 explained later) where two wipers 2 are provided on front and back sides of the outer frame 5B, respectively. Needless to say, the number of wipers 2 is not limited to two but the wiper 2 can be provided on only one of the front and back sides of the outer frame 5B (which case corresponds to FIGS. 1 to 7).

With the depilating device thus configured, the height of the wiper 2 can be switched only by operating the height switching button 29. Due to this, if soap is used to remove hair, the wiper 2 can be held at a position at which the wiper 2 strikes against the skin surface 6 by lifting the wiper 2. At this time, by vertically adjusting the wiper 2, it is possible to easily adjust pressing load of the wiper 2 on the skin surface 6, whereby the stimulus can be made weaker. Besides, it is possible to remove hair while stretching the skin using the wiper 2 by the appropriate pressing load of the wiper 2 on the skin surface 6. On the other hand, if the depilating device is used without soap, the wiper 2 can be lowered and held at a position at which the wiper 2 does not touch the skin. It is, therefore, possible to easily deal with depilation in a dry state, thereby further improving the user-friendliness of the depilating device.

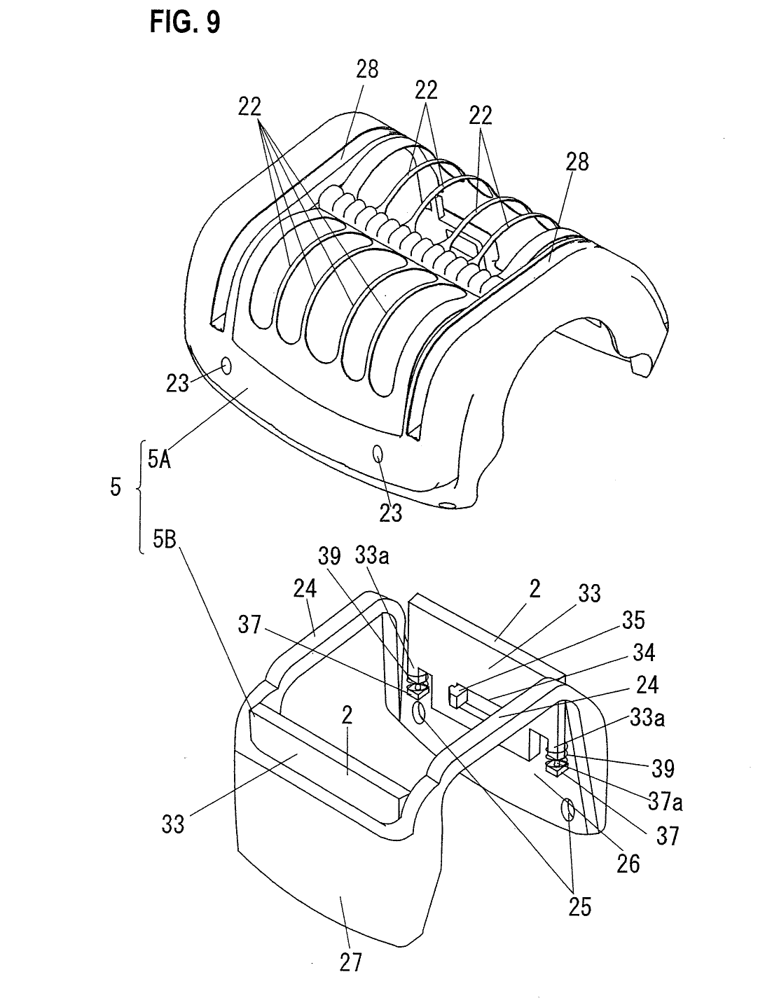

FIG. 9 shows an instance where the wiper 2 is floatable on the skin in a depilating device according to a third embodiment of the present invention. Other constituent elements of the depilating device according to the third embodiment are similar to those according to the first embodiment shown in FIGS. 5 and 6. In FIG. 9, like or corresponding constituent elements are denoted by the same reference symbols as those according to the first embodiment. In the third embodiment, the lifting plate 33 including the wiper 2 is vertically slidably supported along an inner surface of the frame 5B by a retaining hook 35 provided on the inner surface of the outer frame 5B to protrude therefrom while the wiper 2 is being retained by the retaining hook 35. The retaining hook 35 is engaged with the lateral bore 34 formed in the lifting plate 33. In the third embodiment, the lateral bore 34 of the lifting plate 33 is formed to have a size enough to be able to move the retaining hook 35 upward and downward. Legs 33a are provided on lower left and right ends of the lifting plate 33, respectively. Spring bearings 37 each having a protrusion 37a are provided on the inner surface of the outer frame 5B to protrude therefrom. Each of the legs 33a of the lifting plate 33 is supported by each spring bearing 37 having the protrusion 37a via a floating spring 39. By so configuring, the lifting plate 33 including the wiper 2 is made floatable by the floating springs 39 in a direction in which the wiper 2 is close to or apart from the skin. It is, therefore, possible to prevent the wiper 2 from striking against the skin strongly and eliminate the stimulus of the wiper 2 on the skin. Furthermore, according to the third embodiment, the wiper 2 is made deformable by the floating springs 39 in a direction almost perpendicular to the skin surface 6. It is, therefore, possible to considerably improve the follow-up performance of the wiper 2 even with respect to irregular parts of the skin surface or such a convex part as a bony part of the skin surface. It is thereby possible to ensure wiping off soap bubbles on whichever part of the skin surface 6.

FIG. 10 shows an instance where wipers 2 are provided on both the front side F and the back side D of the main body casing 41, respectively in a depilating device according to a fourth embodiment of the present invention. In this case, it is possible to attain the wiping effect produced by the wipers 2 whether the main body casing 41 is moved in either direction of the front side F or the back side D. In the fourth embodiment, the depilating device is preferably structured to include a tank for storing soapy water in the main body casing 41 so as to supply the soapy water to the depilation head 1H.

By providing the wiper for wiping off soap bubbles adhering to the skin surface after depilating on the head frame surrounding the depilating portion, the wiper wipes off the bubbles adhering to the skin surface after depilating when the depilation is made using soap. Therefore, it is possible to attain the depilating device that makes it possible to easily recognize unremoved hairs, that saves time and labor for wiping off the bubbles, and that can ensure quite high user-friendliness while making safe and smooth depilation using the soap.

While the embodiment of the present invention has been described above, the invention is not limited to the above embodiment and changes and modifications can be made within the scope of the gist of the present invention.

What is claimed is:

1. A depilating device comprising:
a main body casing including a manually grippable grip and having a waterproof structure;
a depilation head including a depilating portion for removing hair and a head frame surrounding the depilating portion, the depilation head attached to the main body casing; and
a wiper provided on the head frame, and wiping off a soap bubble adhering to a skin surface after depilating by the depilating portion.

2. The depilating device according to claim 1, wherein the wiper is plate-shaped to extend almost in parallel to the skin surface while the depilation head is pressed against the skin surface.

3. The depilating device according to claim 1, wherein the wiper is deformable in a direction almost perpendicular to the skin surface.

4. The depilating device according to claim 1, wherein the wiper is floatable on the skin surface.

5. The depilating device according to claim 1, wherein the wiper is provided on one of a front side and a back side of the main body casing.

6. The depilating device according to claim 1, wherein the wiper and the head frame are detachable from the main body casing.

7. The depilating device according to claim 1, wherein the wiper is detachable from the head frame.

8. The depilating device according to claim 1, wherein the wiper is height-adjustable with respect to the head frame in a direction almost perpendicular to the skin surface.

9. The depilating device according to claim 1, wherein the wiper is made of elastomer.
